(19) United States
(12) Patent Application Publication
(10) Pub. No.: US 2008/0274019 A1
(43) Pub. Date: Nov. 6, 2008

(54) Preparation of Derivative of Polyhydric Alcohols

(76) Inventors: Joseph Robert Beggin, Warrensburg, IL (US); Thomas P. Binder, Decatur, IL (US); Ahmad K. Hilaly, Springfield, IL (US); Lawrence P. Karcher, Decatur, IL (US); Brad Zenthoefer, Decatur, IL (US)

Correspondence Address: ARCHER DANIELS MIDLAND COMPANY, 4666 FARIES PARKWAY, DECATUR, IL, 62526, US

(21) Appl. No.: 12/108,060

(22) Filed: Apr. 23, 2008

Related U.S. Application Data
(60) Provisional application No. 60/913,572, filed on Apr. 24, 2007.

(51) Int. Cl. B01J 19/00; C07C 29/00; C07C 29/74

(52) U.S. Cl. 422/129; 568/852; 568/868

(57) ABSTRACT

A method for converting a polyhydric alcohol into propylene glycol and butanediols is disclosed. Also disclosed are methods for converting polyhydric alcohols into three-carbon products and four-carbon products. Also disclosed are methods for maximizing conversion of polyhydric alcohols and minimizing formation of reaction products that are difficult to remove from the desired product. In other embodiments, methods are described to optimize use of reactants, including hydrogen, in hydrogenolysis of polyhydric alcohols.

### CROSS-REFERENCE TO RELATED APPLICATIONS

This application claims the benefit of U.S. Provisional Application No. 60/913,572, filed Apr. 24, 2007, the contents of the entirety of which are incorporated by this reference.

### TECHNICAL FIELD

This teaching relates to a process for adding value to a bio-based feed stock such as for example glycerol, which is obtained from processing of fats, oils and soap-stock. An alternative feedstock, sorbitol, can be obtained as a product of hydrogenation of glucose from starch.

### BACKGROUND OF THE INVENTION

Many chemicals produced industrially are obtained from petroleum and natural gas based sources. High prices of this raw material in additional to limited availability, and environmental consequences surrounding the extraction, transportation and refining of petroleum compounds into industrial chemicals have shown a need for developing such products from bio-based or renewable sources. Bio-based feedstocks such as corn starch or vegetable oils can be obtained from plants and may be subsequently processed through biological processes such as fermentation.

For complete utilization, it is important to convert products obtained from processing of bio-based products into value added chemicals. For instance, in the production of fatty acid methyl esters from vegetable oil about 1 kg of crude glycerol by-product is formed for every 9 kg of biodiesel produced.

Propylene glycol is a three-carbon compound currently derived from petrochemical natural gas. Propylene glycol is produced by hydration of propylene oxide derived from propylene by either the chlorohydrin process or the hydroperoxide process and is a major commodity chemical with an annual production of over 1 billion pounds in the US. Although natural gas is an abundant resource, it is non-renewable.

There are several reports in the literature for the production of propylene glycol from renewable feed stocks. Most commonly, they involve hydrogenolysis of sugars or sugar alcohols at high temperatures and pressures in the presence of a metal catalyst producing propylene glycol and other lower diols.

By-product glycerol can be converted to propylene glycol. The overall reaction scheme for converting glycerol to glycerol derivatives is given below.

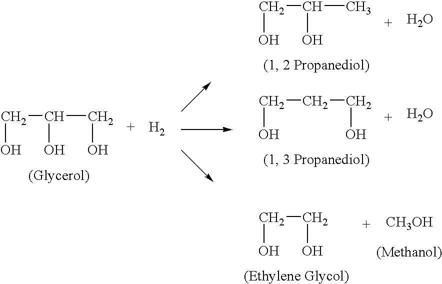

In the presence of gaseous hydrogen and metallic catalysts, sorbitol (a polyhydric alcohol) or glycerol (a polyhydric alcohol) can be hydrogenated to propylene glycol (a three-carbon compound); 1,3 propanediol (a three-carbon compound); or ethylene glycol (a two-carbon compound) or methanol (a one-carbon compound).

Processes for hydrogenating glycerol using copper and zinc catalysts in addition to a sulfided ruthenium catalyst at pressures over 2100 psi and temperatures between 240-270° C. are described in U.S. Pat. Nos. 5,276,181 and 5,214,219. A process of preparing 1,2 propanediol by catalytic hydrogenation of glycerol at elevated temperatures and pressures using a catalyst comprising the metals cobalt, copper, manganese and molybdenum is outlined in U.S. Pat. No. 5,616,817.

However, known processes exhibit poor selectivity and require large amounts of water, which dilute the glycerol feed stock. In order to isolate glycerol derivatives, it is therefore necessary first to remove a large amount of water by distillation which increases the costs of production. Consequently, a need exists in the industry for efficient processes for converting glycerol obtained from fats, oils and soap processing to higher value products such as glycerol derivatives. With the growth of the fatty acid methyl ester (or biodiesel) industry the need to develop value added commodities from this feed stock has grown.

### BRIEF DESCRIPTION OF DRAWINGS

FIG. 1 is a schematic block flow diagram illustrating one teaching of a separator-reactor-separator with a feed purifier, reactor, product purifier and product fractionator described by the present teaching.

FIG. 2 is a schematic block flow diagram of one teaching of a process illustrating the simplified feed purifier, reactor, product purifier and product fractionator of described by the present teaching.

FIG. 3 is a schematic block flow diagram illustrating separator-reactor-separator with a feed purifier, reactor, product purifier and product fractionator and unreacted glycerol recycle stream.

FIG. 4 is a schematic block flow diagram illustrating separator-reactor-separator with a feed purifier, reactor, product purifier and product fractionator and use of a membrane vapor permeation device to recover low concentrations of alcohols present in distillation product.

FIG. 5 is a schematic block flow diagram illustrating separator-reactor-separator with a feed purifier, reactor, product purifier and product fractionator. It also includes an optional bipolar membrane setup for fractionating salt waste to produce acid and base that can be recycled in the process.

FIG. 6 is a schematic block flow diagram illustrating separator-reactor-separator with a feed purifier, reactor, product purifier and product fractionator and use of a pressure swing adsorption of membrane separation device to purify and recycle the excess hydrogen present in reactor product.

FIG. 7 is a schematic block flow diagram illustrating separator-reactor-separator with a feed purifier, reactor, product purifier and product fractionator for separating unreacted glycerol as a recycle stream. It includes the use of a pressure swing adsorption of membrane separation device to purify and recycle the excess hydrogen present in reactor product.

FIG. 8 is a schematic block flow diagram illustrating separator-reactor-separator with a feed purifier, reactor, product purifier and product fractionator for separating unreacted glycerol as a recycle stream. It includes the use of a pressure swing adsorption of membrane separation device to purify and recycle the excess hydrogen present in reactor product. Also a pH adjustment step before product purification is added to enhance the product purification recoveries and yields.

FIG. 9 is a schematic block flow diagram illustrating a membrane separation system suitable for purification of hydrogen off-gas stream from a hydrogenolysis or hydrocracking reactor.

FIG. 10 is a schematic block flow diagram illustrating production of polyhydric alcohols by means of a hydrogenolysis, hydrocracking or any other suitable method as known in the art.

FIGS. 11 and 12 are schematic block flow diagrams illustrating separation of impurities from hydrogen used in the hydrogenolysis reactor to enable recycling of substantially purified hydrogen.

### SUMMARY OF THE INVENTION

In one exemplary method a system for treating glycerol or sorbitol is disclosed. The system may comprise a reactor, a conduit for conducting glycerol or sorbitol to a reactor, a conduit for conducting reaction product to a product purifier, a product purifier and a conduit for conducting product purifier effluent. The system converts glycerol or sorbitol into propylene glycol and butanediol. In certain aspects, the glycerol may be purified.

In another exemplary embodiment, a system for producing propylene glycol is disclosed. In one embodiment, the system may comprise a reactor, reactor product, a product purifier and a product purifier effluent wherein the content of propylene glycol in product purifier effluent is greater than the content of propylene glycol in reactor product. In certain aspects, the reactor product may comprise butanediol.

In another embodiment, a process for converting a polyhydric alcohol into a three-carbon compound and a four-carbon compound is presented. In aspects, the polyhydric alcohol is combined with hydrogen, heated, and placed in contact with a catalyst to form a reaction product. The reaction product is acidified and the three-carbon compound, the four-carbon compound, or combinations thereof are removed. In aspects, the process includes a product purifier.

In a further exemplary embodiment, a process for converting glycerol or sorbitol into a mixture of propylene glycol and butanediols is disclosed. In certain aspects, butanediols may be removed from propylene glycol. In certain aspects, the glycerol may be purified before the process.

In another exemplary embodiment, a process for converting glycerol into a mixture of propylene glycol and butanediol while controlling the content of propylene glycol in a reactor effluent is disclosed. In certain aspects, means for controlling the content of propylene glycol comprise controlling the pH of reactor feedstock, controlling an amount of promoter in a reactor feed, controlling an amount of catalyst in the reactor, controlling the liquid hourly space velocity in a reactor, controlling the weight hourly space velocity in a reactor, controlling hydrogen pressure, and combinations of any thereof.

In an embodiment, a process for converting glycerol into a mixture of propylene glycol and butanediols while controlling the content of butanediol in a reactor effluent is disclosed. In certain aspects, means for controlling the content of butanediol comprise controlling the pH of reactor feedstock, controlling an amount of promoter in a reactor feed, controlling an amount of catalyst in the reactor, controlling the liquid hourly space velocity in a reactor, controlling the weight hourly space velocity in a reactor, controlling hydrogen pressure, and combinations of any.

In certain embodiments, a reactor product may be acidified and propylene glycol, ethylene glycol or a combination thereof may be recovered. In some aspects, the reaction product may be acidified to a pH of between 2 and 8.

In an additional embodiment a method of recycling the hydrogen used in the hydrogenolysis reactor are described. In one embodiment, the recycling may be done with a gas booster. Certain aspects of this embodiment may involve the use of a membrane or pressure swing adsorption device to achieve the desired purity of recycled hydrogen.

In yet another embodiment, a process for converting a polyhydric alcohol into at least one three-carbon compound and at least one four-carbon compound by combining a polyhydric alcohol, a catalyst, and hydrogen in a reactor is presented. In certain aspects, the polyhydric alcohol may be purified. In aspects, the polyhydric alcohol may be purified by ion exclusion, electro-dialysis, filtration, distillation, ion exchange, and combinations thereof.

In other embodiments a method is described for purifying the hydrogenolysis reaction product by first acidifying the product with a mineral acid and then subjecting the product to distillation to recover the desired polyhydric alcohols.

Other embodiments describe a facility configured suitably to perform the disclosures provided herein or products manufactured the methods provided here in. Also disclosed are applications of such products in household, industrial or commercial uses.

### DETAILED DESCRIPTION

Propylene glycol is a three carbon diol with a steriogenic center at the central carbon atom. Propylene glycol is commonly used in a variety of consumer products and food products, including deodorants, pharmaceuticals, moisturizing lotions, and fat-free ice cream and sour cream products. It also finds uses in hydraulic fluids, and as a solvent. Ethylene glycol and propylene glycol are used to make antifreeze and de-icing solutions for cars, airplanes, and boats; to make polyester compounds; and as solvents in the paint and plastics industries. Ethylene glycol, a two-carbon compound, is also an ingredient in photographic developing solutions, hydraulic brake fluids and in inks used in stamp pads, ballpoint pens, and print shops.

The present disclosure is directed towards a method which enables glycerol to be converted with a high selectivity and rate towards the production of glycerol derivatives such as propylene glycol and ethylene glycol. In an embodiment, impure glycerol, such as glycerol by-product from the processes of saponification or transesterification of fats and oils (such as the production of soap or biodiesel, respectively) to be converted at a high rate and good selectivity into glycerol derivatives such as propylene glycol and ethylene glycol. Another embodiment of the present teaching describes a catalytic method of hydrogenating glycerol in order to produce mainly oxygenated compounds having 1-3 carbon atoms, characterized by reaction in a heterogeneous phase wherein glycerol contacts hydrogen and optionally a promoter, such as an alkali, which is able to react with the glycerol in the presence of a metal catalyst at a temperature of at least 100° C. Another embodiment of the present teaching describes a catalytic process of hydrogenating glycerol in order to produce mainly oxygenated compounds having 1-5 carbon atoms. The process is characterized by reaction in a heterogeneous phase wherein glycerol contacts hydrogen and optionally a promoter, such as an alkali, which is able to react with the glycerol in the presence of a metal catalyst at a temperature of at least 100° C. In another teaching this disclosure describes a process for purifying the raw material used in the reaction and also provides for clean up of the glycerol derivatives produced in the reaction. In yet another teaching, the hydrogen used in the reaction is purified and recycled, thus allowing for reduced costs in the manufacturing of the glycerol derivatives.

The present disclosure further teaches a method for purifying glycerol derivatives produced by techniques described herein, including removal of undesirable impurities from, and fractionation of, the glycerol derivatives. In another teaching, a method for removing foulants and catalyst poisons that inhibit the formation of glycerol derivatives and reduce the selectivity and conversion of glycerol is described. In another teaching, a method is described for recycling unconverted hydrogen and glycerol to the reactor substantially free of impurities, resulting in an increased overall yield for the process.

The present disclosure further teaches the use of bio-derived feed stock for synthesis of polyols. Bio-derived polyol feedstocks can be obtained by subjecting sugars or carbohydrates to hydrogenolysis (also called catalytic cracking). In one teaching, sorbitol may be subjected to hydrogenolysis to provide a mixture comprising bio based polyol reactor product, as described herein. Other polysaccharides and polyols suitable for hydrogenolysis include, but are not limited to, glucose (dextrose), sorbitol, mannitol, sucrose, lactose, maltose, alpha-methyl-d-glucoside, pentaacetylglucose, gluconic lactone and any combinations thereof.

According to other teachings, the bio-based polyol feedstock may be obtained as mixed polyols. Natural fibers may be hydrolyzed (producing a hydrolysate) to provide bio-derived polyol feedstock, such as mixtures of polyols. Fibers suitable for this purpose include, but are not limited to, corn fiber from corn wet mills, dry corn gluten feed which contains corn fiber from dry mills, wet corn gluten feed from wet corn mills that do not run dryers, distiller dry grains solubles (DDGS) and Distiller's Grain Solubles (DGS) from dry corn mills, canola hulls, rapeseed hulls, peanut shells, soybean hulls, cottonseed hulls, cocoa hulls, barley hulls, oat hulls, wheat straw, corn stover, rice hulls, starch streams from wheat processing, fiber streams from corn masa plants, edible bean molasses, edible bean fiber, and mixtures of any thereof. Hydrolysates of natural fibers, such as corn fiber, may be enriched in bio-derived polyol feedstock suitable for use as a feedstock in the hydrogenation reaction described herein, including, but not limited to, arabinose, xylose, sucrose, maltose, isomaltose, fructose, mannose, galactose, glucose, and mixtures of any thereof.

According to other teachings, the bio-derived polyol feedstock obtained from hydrolyzed fibers may be subjected to fermentation or acidification. The fermentation process may provide modified bio-derived polyol feed stocks, or may alter the amounts of residues of polysaccharides or polyols obtained from hydrolyzed fibers. After fermentation, a fermentation broth may be obtained and residues of polysaccharides or polyols can be recovered and/or concentrated from the fermentation broth to provide a bio-derived polyol feedstock suitable for hydrogenolysis, as described herein.

According to certain teachings, the hydrogenolysis product may comprise a mixture of propylene glycol and ethylene glycol, along with minor amounts of one or more of methanol (a one-carbon compound), 2-propanol (a three-carbon compound), glycerol, lactic acid (a three-carbon compound), glyceric acid (a three-carbon compound), sodium lactate (a three-carbon compound), sodium glycerate (a three-carbon compound) and combinations of any thereof. Several four-carbon compounds, such as butanediols (BDO) including 1,2-butanediol, 1,3-butanediol, 1,4-butanediol, and 2,3-butanediol are produced, in addition to five-carbon compounds such as 2,4-pentanediol (2,4-PeDO).

Hydrogenolysis of bio-derived polyol feed stocks includes, but is not limited to, polyol feed stocks derived from biological or botanical sources. For example, bio-derived polyols suitable for use according to various teachings of the present disclosure include, but are not limited to, saccharides, such as, but not limited to, biologically derived (bio-derived) polyols including monosaccharides including dioses, such as glyceraldehydes; trioses, such as glyceraldehyde and dihydroxyacetone; tetroeses, such as erythrose and threose; aldo-pentoses such as arabinose, lyxose, ribose, deoxyribose, xylose; keto-pentoses, such as ribulose and xylulose; aldo-hexoses such as allose, altrose, galactose, glucose (dextrose), gulose, idose, mannose, talose; keto-hexoses, such as fructose, psicose, sorbose, tagatose; heptoses, such as mannoheptulose and sedoheptulose; octoses, such as octolose and 2-keto-3-deoxy-manno-octonate; and nonoses, such as sialose; disaccharides including sucrose (table sugar, cane sugar, saccharose, or beet sugar), including glucose+fructose; lactose (milk sugar) comprising glucose+galactose; maltose (produced during the malting of barley) comprising glucose+glucose; trehalose is present in fungi and insects, is also glucose+glucose; cellobiose is another of the glucose+glucose disaccharides; oligosaccharides, such as raffinose (melitose), stachycose, and verbascose; sorbitol, glycerol, sorbitan, isosorbide, hydroxymethyl furfural, polyglycerols, plant fiber hydrolysates, fermentation products from plant fiber hydrolysates, and various mixtures of any thereof.

The process described herein advances the art for converting glycerol to glycerol derivates and overcomes the problems of the prior art by producing value-added products such as 1,3 propane-diol and ethylene-diol by hydrogenation of bio-derived glycerol feed stocks. In one teaching, the feed stocks include water or a non-aqueous solvent. For instance in certain cases feed containing glycerol, water and a solvent may be used. Non-aqueous solvents that may be used include, but are not limited to, methanol, ethanol, ethylene glycol, propylene glycol, n-propanol and iso-propanol. The feed stocks for this process are commercially available and can also be obtained as byproducts of commercial biodiesel processing. For instance, the feed stocks may be obtained through fats and oils processing or generated as a byproduct in the manufacture of soaps. The feedstock may for example, be provided as glycerol byproduct of primary alcohol alcoholysis of a glyceride, such as a mono-, di- or tri glyceride. These glycerides may be obtained from refining edible and non edible plant feed stocks such as soybeans, canola, corn, rapeseed, palm fruit, flaxseed, wheat germ, rice bran, sunflower, safflower, cotton, peanuts, jatropha and combinations of any thereof. The feed stocks are commonly known to those skilled in the art and can be used either in pure or crude form. Crude glycerin may be contain between 10-90% by weight of glycerol, the remainder comprising other constituents such as water, triglycerides, free fatty acids, soap stock and other non saponifiables. These materials may inhibit or poison the catalyst used for hydrogenolysis of glycerol to prepare the derivates of glycerol.

This disclosure teaches various routes for preparing crude glycerol for hydrogenolysis and also describes methods for purifying glycerol derivatives upon hydrogenolysis. In some embodiments, feed stocks contain 20-80% by weight of glycerol, while the balance includes other components. In some embodiments, the purification steps may be omitted when USP grade glycerol may be used.

Catalysts for the hydrogenolysis processes are solid or heterogeneous catalysis. The catalysts may include those known in the art or as described herein. The catalysts are provided with a high surface area support material that prevents degradation under the reaction conditions. These supports may include, but are not limited to, carbon, alumina, titania and zirconia or any combinations thereof. These supports can also be prepared in mixed or layered materials such as mixed with catalyst materials.

The temperature used in the hydrogenolysis reaction may range from 150° C. to 300° C. while the pressure is between 500 psi and 2000 psi, or 1000 psi to 1600 psi. Reaction time for the hydrogenolysis is defined by the term weight hourly space velocity (WHSV) which is weight of reactant per unit weight of catalyst per hour. Alternatively, the term liquid hourly space velocity (LHSV) may also be used and is defined as volume of reactant per unit volume of catalyst per hour. Ranges for WHSV and LHSV are between 0.1 and 3.0 which can be modified suitably to meet reactor design specifications using techniques well known to those in the art. The selectivity of the catalyst and the yield of PG can be improved by neutralizing the reactant mixture, or by rendering it alkali before or during the hydrogenolysis and carrying out the reaction under alkaline conditions. The reaction may be conducted under basic conditions, such as at pH 8 to 14, or a pH of 10 to 13. The desired pH may be obtained by adding an alkali, such as sodium hydroxide, potassium hydroxide or an alkoxide such as sodium methoxide or potassium methoxide.

In various teachings, alkali may be added to a level of 0.2 to 0.7%. Organic acids formed during hydrogenolysis cause the pH to decrease and the selectivity of the catalyst decreases. Consequently, the reaction is carried out in sufficient alkalinity to ameliorate this problem. The catalyst used in the step of reacting may be a primary heterogeneous catalyst selected from a group comprising palladium, rhenium, nickel, rhodium, copper, zinc, chromium or any combinations thereof. In various teachings, a secondary catalyst may be used in addition to the primary metal catalyst. Additional metals may include, but are not limited to, Ni, Pd, Ru, Co, Ag, Au, Rh, Pt, Ir, Os and Cu. Combinations such as Ni/Re, Cu/Re and Co/Re may be employed. Also, as known by those of ordinary skill in the art, the catalyst may be a homogenous catalyst such as an ionic liquid or an osmonium salt which is a liquid under reaction conditions.

In one embodiment, a route for converting crude glycerol into substantially pure propylene glycol is disclosed. The processes involved in the hydrogenolysis of glycerol may be carried out by any of the routes known by those of ordinary skill in the art. These include heterogeneous metal catalysts such as those described in U.S. Pat. No. 6,479,713, WO2005/051874, US2005/024431, or homogenous catalysts as referred to in the publication Hydrocarbon Processing (February 2006) pp 87-92 (incorporated herein by reference).

In another embodiment, a process for converting a polyhydric alcohol into at least one three-carbon compound and at least one four-carbon compound is described.

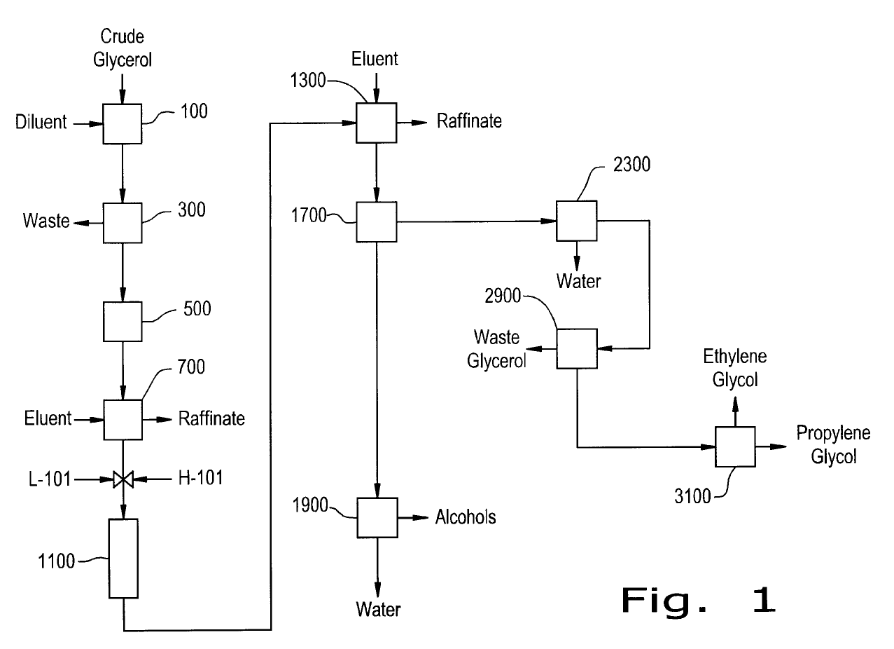

FIG. 1 illustrates a block flow diagram of a process for converting crude glycerol into highly pure PG. Reference number labels in all figures indicate the same feature throughout this description and the figures. For example, label 1100 refers to a reactor throughout this specification. Crude glycerol is mixed with a diluent in mixer type equipment 100. The diluent could be an alcohol or water. The crude glycerol solution is processed by a gravity separation device 300 such as a centrifuge or hydrocylone which removes heavier impurities and sediments and provides a purer glycerol solution. The supernatant from gravity separation 300 is treated with an adsorption bed 500 which could be a carbon or resin adsorbent bed. Several types of carbon and resins are well known to those of ordinary skill the art. For instance, carbon types of CPG 12×40™ or CPG 20×50™ or CAL TR™ from Calgon Carbon Corporation (Pittsburgh, Pa.) can be used. Alternative carbon types include Optipore SD-2™ or similar type of resin from Rohm and Haas Inc. (Philadelphia Pa.) may also be used.

The adsorption step removes organic impurities that are present in the glycerol solution and improves the performance of the conversion steps required for hydrogenolysis of glycerol. The stream from adsorption step 500 is treated in a chromatography step 700 using ion exclusion or ion exchange type chromatography using resins such as UBK 555™ in Na⁺ form (available from Mitsubishi Chemical Corporation Tokyo Japan). Activated charcoal such as CAL TR™ or CPG™ (available from Calgon Carbon, Pittsburg, Pa.) may also be used. Such processes involve using an eluent such as water to remove the charged impurities present in the glycerol solution. Simulated moving bed chromatography, such as with a C-SEP, provides suitable purification. The resulting glycerol solution or other polyhydric feed is stepwise or continuously introduced through a conduit into reactor 1100. Hydrogen is added to hydrogen line H-101 and pH modifier is introduced to feed line L-101 to promote conversion of glycerol to PG. The reactor may be as described in the art or based on the teachings of this disclosure.

The reaction product from the reactor 1100 is introduced into a product purifier (purification device) 1300 through a conduit for conducting reaction product to a product purifier. Product purifier 1300 may be similar to the one described in step 700. An ion exclusion or mixed bed ion exchange device is used to remove the excess pH modifier introduced in step L-101 and also to remove any impurities such as organic acids that are generated in step 1100. The product is subjected to distillation 1700 wherein low molecular weight components such as alcohols and water are removed by vaporization and passed through a conduit for conducting product purifier effluent for further processing in a secondary distillation 1900 to separate the alcohols from water. Distillation bottoms from 1700 are passed through a conduit for conducting product purifier effluent for processing through a series of small distillation columns 2300 and 2900 wherein water and waste glycerol are separated. Reactor product enriched in propylene glycol is passed through a conduit for conducting product purifier effluent to column 3100 to separate purified propylene glycol and ethylene glycol.

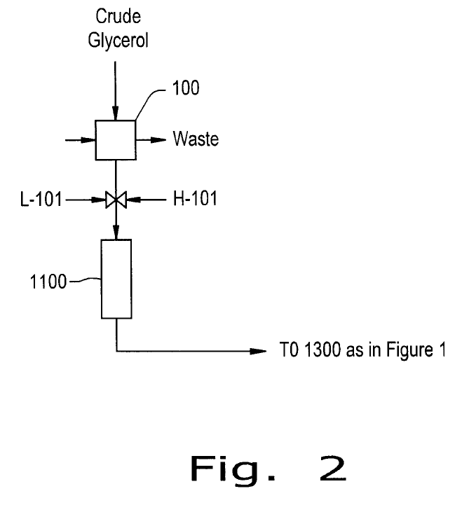

An alternate scheme for the process is shown in FIG. 2, which is a modification of part of FIG. 1, wherein crude glycerol is treated in distillation column 100 to remove the waste salt and organic impurities and processed through steps 1100-3100 as described herein.

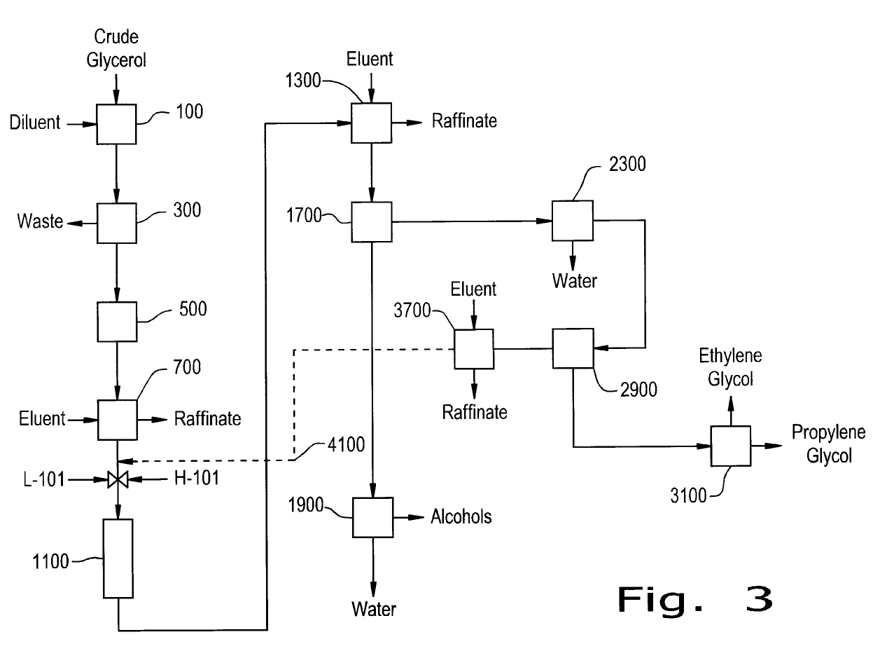

Another alternate scheme, which is a modification of FIG. 1, is shown in FIG. 3 where in unconverted glycerol in a product from separator 2300 is purified in ion exclusion or mixed bed ion exchange type equipment 3700 and recycled back to the reactor 1100. This scheme allows the reactor 1100 to run at higher WHSV and convert less of the feed while allowing for the unconverted glycerol to be recycled through line (conduit) 4100.

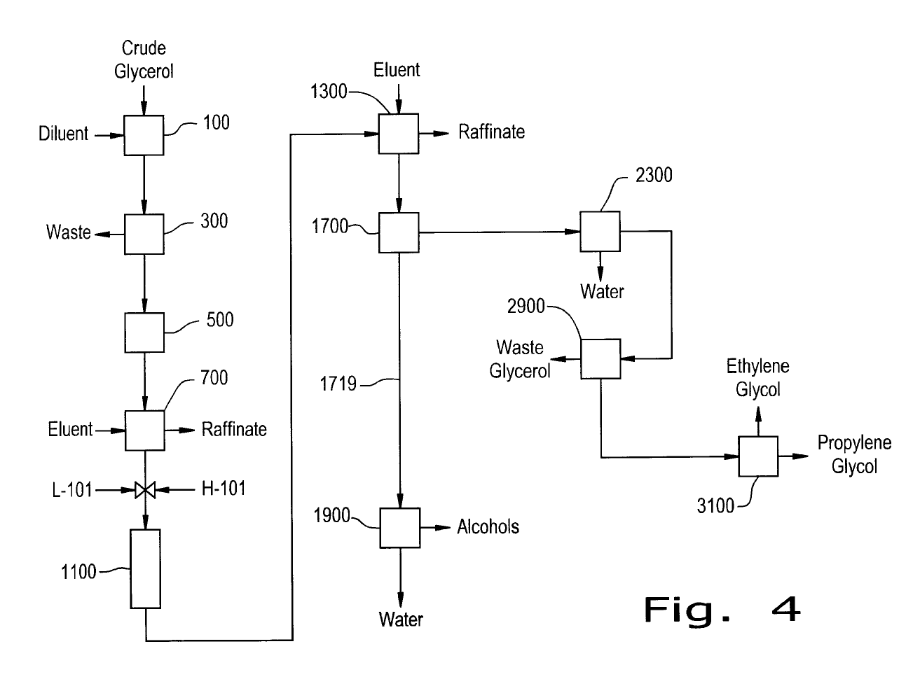

Another alternate scheme, which is a modification of FIG. 1, is shown in FIG. 4 and allows for a vapor permeation type membrane to be used in step 1900 to separate the alcohols from water. This is because the stream 1719 may contain low concentrations of alcohols (less than 10%, mostly 1-2%) which are difficult to recover using distillation. Consequently, a vapor permeation membrane is employed to recover the dilute alcohol stream from step 1900.

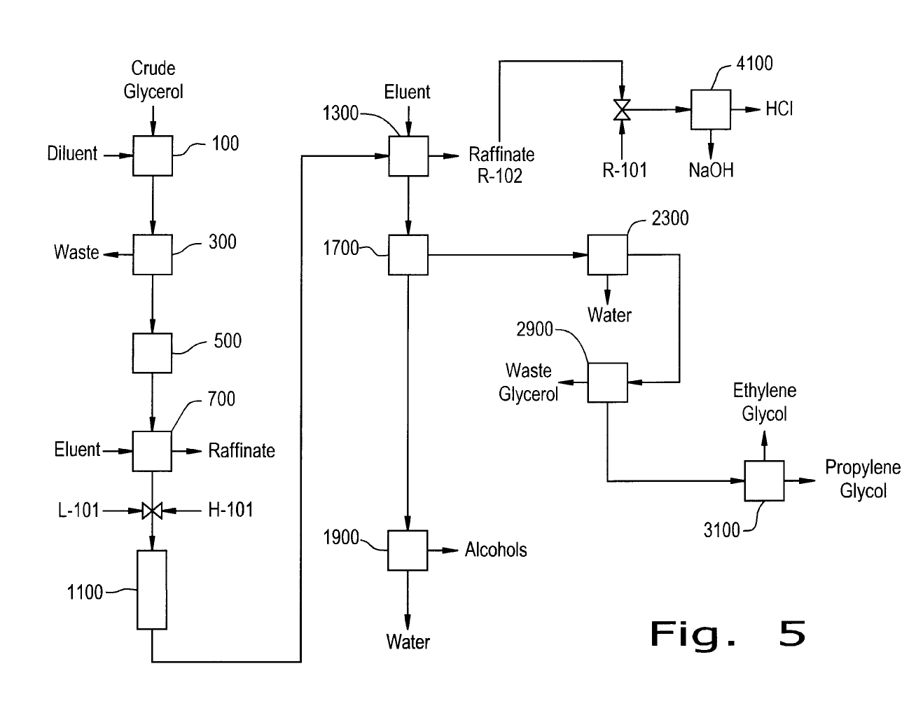

Another alternate scheme, which is a modification of FIG. 1, is shown in FIG. 5 and allows for the waste salt removed in steps R-101 and R-102 to be converted into acid and base using bipolar electrodialysis in unit 4100.

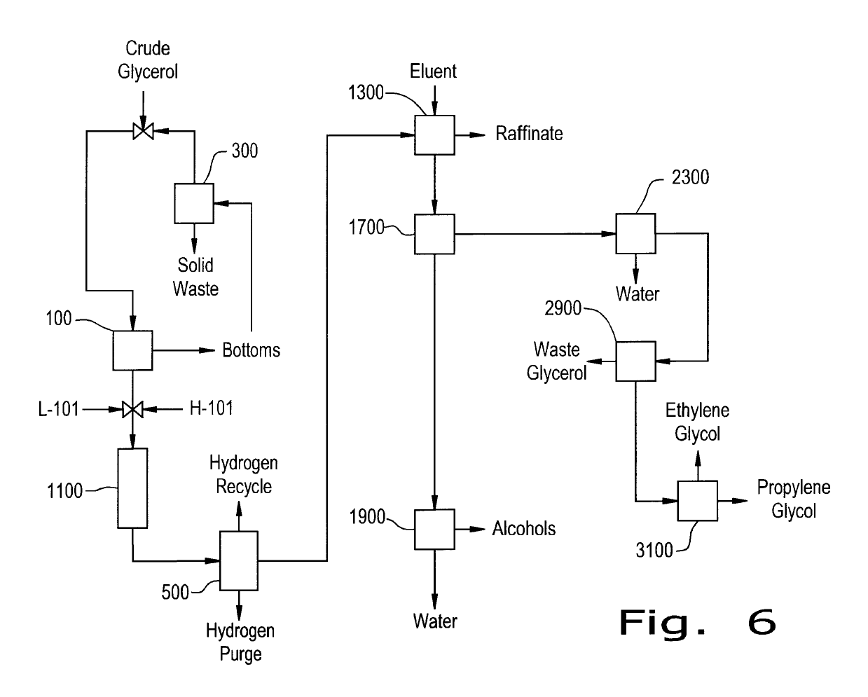

FIG. 6 illustrates a block flow diagram of process for converting crude glycerol into highly pure PG. Crude glycerol is distilled in a still 100 to recover substantially purified glycerol. The bottoms of the still 100 are recycled to a thin film evaporator 300 to recover a solid salt waste and evaporated glycerol which is mixed with the feed going to distillation column 100. The resulting glycerol solution or other polyhydric feed is stepwise or continuously introduced into reactor 1100. Hydrogen is added to hydrogen line H-101 and pH modifier is introduced to feed line L-101 to promote conversion of glycerol to PG. The reactor may be as described in the art or based on the teachings of this disclosure.

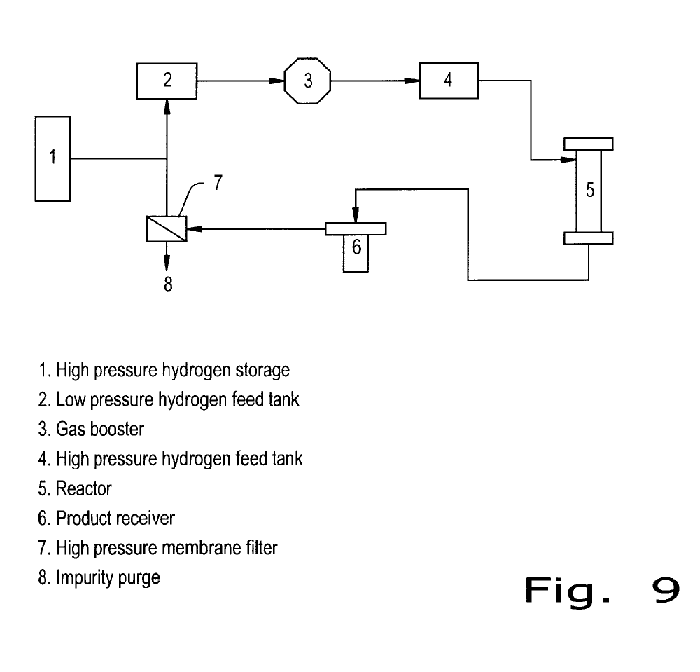

Unconverted hydrogen from the reactor 1100 is purified using a gas separation device 500 to recycle substantially pure hydrogen stream which is processed through a gas booster (FIG. 9) to increase its pressure for reuse in the reactor. A portion of impurities is purged that may be used else where in the process (for instance in boilers for energy or steam generation). The gas separation is a dense membrane type or pressure swing adsorption type that allows for 99% or higher purity hydrogen to be recycled with very little loss in pressure. The product from the reactor 1100 (FIG. 6) contains propylene glycol product and is introduced into a purification device 1300 which may be similar to the one described in step 700. An ion exclusion or mixed bed ion exchange device is used to remove the excess pH modifier introduced in step L-101 and also remove any impurities such as organic acids that are generated in step 1100. The product, containing propylene glycol and ethylene glycol, is subjected to distillation 1700 wherein low molecular weight components such as alcohols and water are removed by vaporization and further processed in a secondary distillation 1900 to separate the alcohols from water. Distillation bottoms from 1700, containing propylene glycol and ethylene glycol, are processed through a series of small distillation columns 2300 and 2900 wherein water and waste glycerol are separated, followed by fractionation of glycerol derivates in column 3100 to separate purified propylene glycol and ethylene glycol.

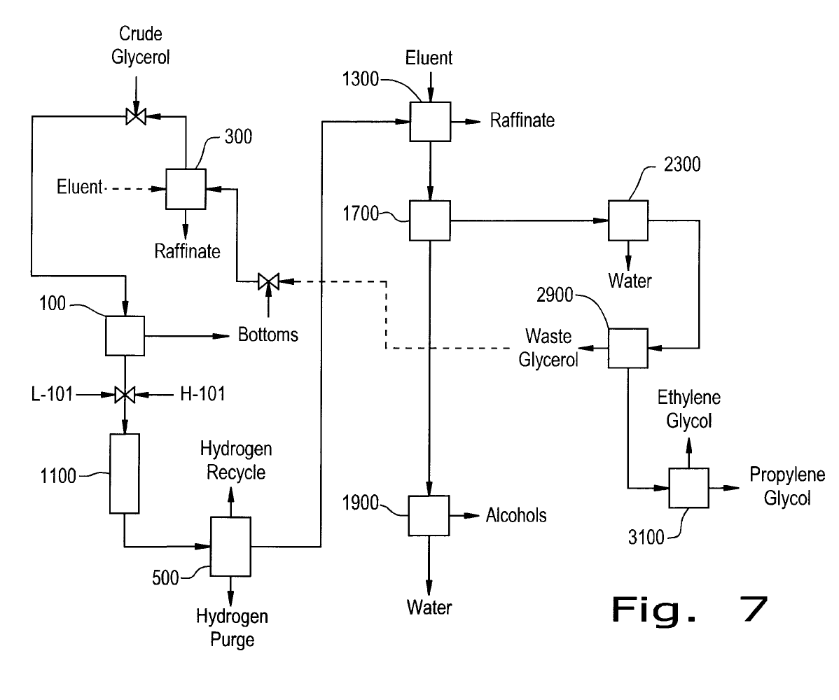

FIG. 7 is a modification of FIG. 6 and provides a block flow diagram of process for converting crude glycerol into highly pure PG. Crude glycerol is distilled in still 100 to recover substantially purified glycerol. The bottoms of the still 100 are recycled to a thin film evaporator 300 to recover a solid salt waste and evaporated glycerol which is mixed with the feed going to distillation column 100. The resulting glycerol solution or other polyhydric feed is stepwise or continuously introduced into reactor 1100. Hydrogen is added to hydrogen line H-101 and pH modifier is introduced to feed line L-101 to promote conversion of glycerol to PG. The reactor may be as described in the art or based on the teachings of this disclosure.

Unconverted hydrogen from the reactor 1100 is purified using a gas separation device 500 to recycle substantially pure hydrogen stream which is processed through a gas booster to increase hydrogen pressure for reuse in the reactor A portion of impurities is purged that may be used else where in the process (for instance in boilers for energy or steam generation). The gas separation is a dense gas membrane type or pressure swing adsorption type that allows for 99% or higher purity hydrogen to be recycled with very little loss in pressure. The product from the reactor 1100, containing propylene glycol and ethylene glycol, is introduced into a purification device 1300 which may be similar to the one described in step 700. An ion exclusion or mixed bed ion exchange device is used to remove the excess pH modifier introduced in step L-101 and also remove any impurities such as organic acids that are generated by step 1100. The product, containing propylene glycol and ethylene glycol, is subjected to distillation 1700 wherein low molecular weight components such as alcohols and water are removed by vaporization and further processed in a secondary distillation 1900 to separate the alcohols from water. Distillation bottoms from 1700 are processed through a series of small distillation columns 2300 and 2900 wherein water and waste glycerol are separated followed by fractionation of glycerol derivates in column 3100 to separate purified propylene glycol and ethylene glycol. The waste glycerol from step 2900 is recycled back to thin film evaporator 100 to enhance the conversion of feed material.

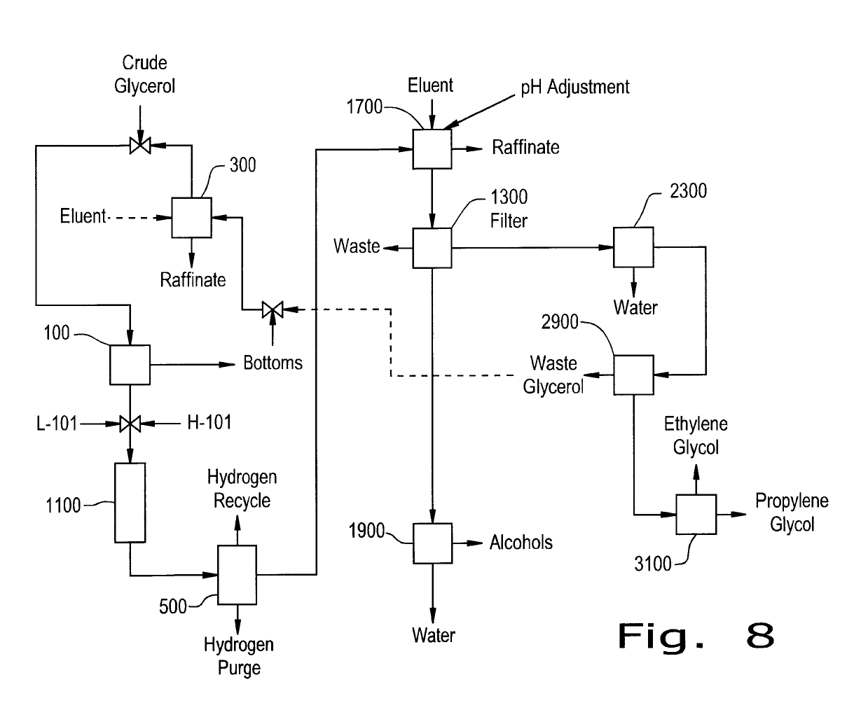

FIG. 8 is a modification of FIG. 7 and provides a block flow diagram of process for converting crude glycerol into highly pure PG. Crude glycerol is distilled in still 100 to recover substantially purified glycerol. The bottoms of the still 100 are recycled to a thin film evaporator 300 to recover a solid salt waste and evaporated glycerol which is mixed with the feed going to distillation column 100. The resulting glycerol solution or other polyhydric feed is stepwise or continuously introduced into reactor 1100. Hydrogen is added to hydrogen line H-101 and pH modifier is introduced to feed line L-101 to promote conversion of glycerol to PG. The reactor may be as described in the art or based on the teachings of this disclosure. Unconverted hydrogen from the reactor 1100 is purified using a gas separation device 500 to recycle substantially pure hydrogen stream which is processed through a gas booster to increase its pressure for reuse in the reactor. A portion of impurities is purged that may be used else where in the process (for instance in boilers for energy or steam generation). The gas separation is a dense membrane type or pressure swing adsorption type that allows for 99% or higher purity hydrogen to be recycled with very little loss in pressure. The product from the reactor after hydrogen recycling (step 500) can be alternatively pH modified to form salts or alkali used in the reactor. The pH modification may be achieved using a suitable acid for example a mineral or organic acids such sulfuric acid or citric acid. The product is subjected to distillation 1700 wherein low molecular weight components such as alcohols and water are removed by vaporization. The pH modification prior to step 1700 followed by filtration step 1300 and distillation steps (2300, 2900 and 3100) reduces or prevents the polymerization of glycerol and degradation of propylene glycol during distillation steps 2300, 2900 and 3100. The product from distillation column 1700 is filtered to remove particulate and suspended impurities in step 1300. This results in increased yield of PG through the process. The product from distillation column 1700 is further processed in a secondary distillation 1900 to separate the alcohols from water. Distillation bottoms from 1700 are processed through a series of distillation columns 2300 and 2900 wherein water and waste glycerol are separated followed by fractionation of glycerol derivates in column 3100 to separate purified propylene glycol and ethylene glycol. The waste glycerol from step 2900 is recycled back to thin film evaporator 100 to enhance the conversion of feed material.

The hydrogen purification system 500 in FIGS. 6-8 may include a membrane system as one method to affect the separation. Such a system is described in FIG. 9. The gas is contacted with a membrane (7), wherein the membrane is of a material and construction that allows small molecules like hydrogen to pass through (permeate) while the larger molecules (such as alkanes and alcohols and other organic products, collectively) do not permeate.

In another teaching of this disclosure, large molecules permeate through a membrane and small molecules such as hydrogen do not. Membranes are a cost effective alternative to, for example, a pressure swing absorption unit. The membranes typically reduce the pressure of the product hydrogen so it has to be compressed prior to use. However, the pressure of the non-permeate is sufficiently high to allow use in a combustion turbine without further compression. The effluent gas from a pressure-swing absorption unit is provided at nearly atmospheric pressure, and subsequent utilization for any application other than boiler fuel requires compression. The membrane can be of any type which allows for permeation of hydrogen gas over carbon dioxide and carbon monoxide. Many types of membrane materials are known in the art which are selective for diffusion of hydrogen compared to nitrogen. Such membrane materials include those including silicon rubber, butyl rubber, polycarbonate, poly (phenylene oxide), nylon 6, 6, polystyrenes, polysulfones, polyamides, polyimides, polyethers, polyarylene oxides, polyurethanes, polyesters, and the like. The membrane units may be of any conventional construction, and a hollow fiber type construction may be used. A hydrogen enriched permeate gas containing between about 30 and 100, typically about 99, mole percent hydrogen and between about 0.1 and about 70, typically about 0.5, mole percent total of alkanes, alcohols and organic acids, permeates through the membrane. The permeate experiences a substantial pressure drop of between about 300 to 700 psi, typically 500 to 700 psi, as it passes through the membrane.

The hydrogen-rich permeate is compressed to between about 800 and 2000 psi for use in subsequent operations. Power for compression may be obtained by the partial expansion of the non-permeate. The non-permeate is advantageously burned in a combustion turbine to generate power. Combustion turbines typically operate with feed pressure of between about 200 psi and 500 psi.

The non-permeate gas stream from the membrane, in line (8) in FIG. 9, contains alkanes, alcohols, and some hydrogen. This non-permeate gas is at high pressure. The non permeating streams pressure is virtually unaffected by the membrane. While this non-permeate gas may be burned in boilers or other heat generating processes, this gas is burned in a combustion turbine to generate power. In another teaching of this disclosure, if the membrane permeates the impurities and allows substantially pure hydrogen to be retained. The permeate stream in this case is purged and may be burned in boilers or other heat generating processes, this gas is advantageously burned in a combustion turbine to generate power.

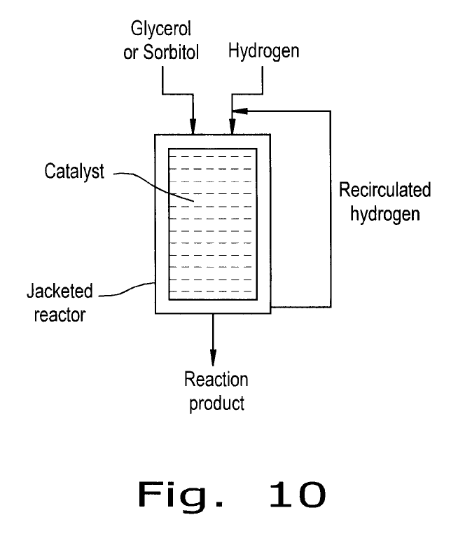

FIG. 10 depicts the reactor process when sorbitol, purified glycerol, or other pure polyols are contacted with hydrogen and catalyst in a reactor to produce a reactor product (reaction product).

### EXAMPLES

### Example 1

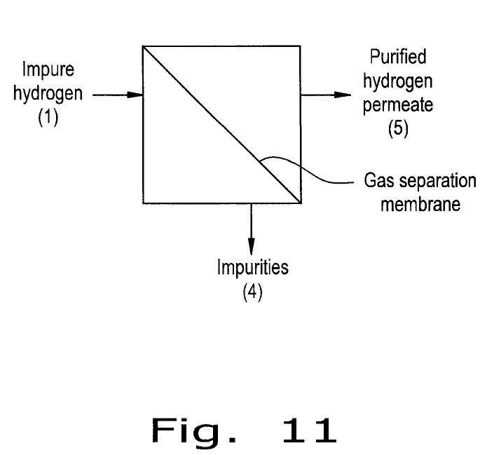

A feed stream (Table 1, Column labeled 1) containing 98% hydrogen and 2% impurities was treated with a PDMS based hydrophobic dense gas separation membrane as depicted in FIG. 11. The hydrogen feed stream was allowed to go through the membrane, which retained the impurities. A permeate stream (Table 1, Column labeled 4) containing 99.29% pure hydrogen was recovered with over 86.6% yield and a pressure drop of only 1.57%. This hydrogen was suitable for use in reactions of the present disclosure. A retentate stream enriched in impurities was obtained (Table 1, Column labeled 5).

### Example 2

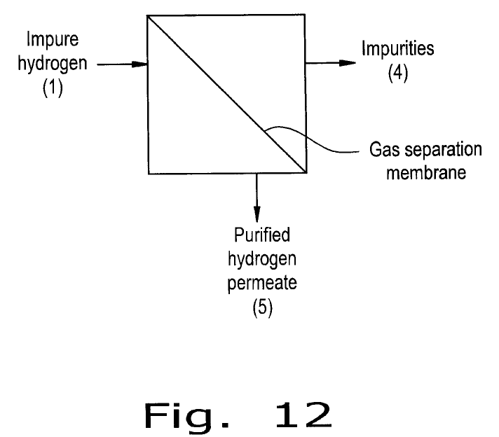

A feed stream (Table 2, Column labeled 1) containing 98% hydrogen and 2% impurities was treated with a polymer based reverse-selective dense gas separation membrane (hydrogen rejecting membrane) as depicted in FIG. 12. Impurities passed go through the membrane as a permeate stream (Table 2, Column labeled 4). A retentate stream (Table 2, Column labeled 5) containing 98.6% pure hydrogen was retained and recovered with over 63.34% yield and a pressure drop of only 4.33%. This hydrogen was suitable for use in reactions of the present disclosure.

### Example 3

A series of studies were conducted in a 2000 ml high-pressure Stainless Steel 316 reactor. As described in FIG. 10, a solid catalyst was loaded in the reactor to a final volume of 1000 ml of catalyst. The reactor was jacketed with a hot oil bath to provide for the elevated temperature for reactions and the feed and hydrogen lines were also preheated to the reactor temperature. A solution of pure glycerol was fed through the catalyst bed at LHSV ranging from 0.5 hr⁻¹ to 2.5 hr⁻¹. Hydrogen was supplied at 1200-1600 psi and was also re-circulated through the reactor at a hydrogen to glycerol feed molar ratio of 1:1 to 10:1, such as at 5:1.

Table 4 describes the results with hydrogenolysis of 40% USP grade glycerol feed. Between 47.7-96.4% of the three-carbon compound glycerol was converted and between 36.3-55.4% of the three-carbon compound propylene glycol was recovered. In addition to propylene glycol, the reaction product contained 0.04-2.31% of the four-carbon butanediol compounds and other non-PG diols, which were recovered from the reaction product (Table 3).

### Example 4

Examples 4-7 describe methods to reduce the formation of four-carbon product BDO and maximize the conversion of polyhydric alcohol glycerol to three-carbon product propylene glycol with a solid phase catalyst such as the “G” catalyst as disclosed in U.S. Pat. No. 6,479,713 or the “HC-1” catalyst available from Sud Chemie (Louisville, Ky.). Hydrogenolysis of a 40% solution of glycerol was carried out substantially as described in Example 3. The effect of the concentration of alkali (sodium hydroxide) in the feed at constant temperature and constant LHSV on the amount of BDO formed was investigated. Higher levels of sodium hydroxide resulted in greater formation of BDOs, thus, the formation of BDOs was minimized when the reaction was operated at lower concentrations (1-1.9 wt %) of alkali promoter (Table 4).

### Example 5

Hydrogenolysis of a 40% solution of the polyhydric alcohol glycerol was carried out substantially as described in Example 3. The effect of the reaction temperature at constant concentrations of alkali (sodium hydroxide) and constant LHSV on the amount of BDO formed was investigated. Higher temperatures resulted in greater formation of BDOs, thus the formation of BDOs was minimized when the reaction was operated at lower reaction temperatures (178-205° C., Table 4).

### Example 6

Hydrogenolysis of a 40% solution of the polyhydric alcohol glycerol was carried out substantially as described in Example 3. The effect of LHSV of the feed at constant concentration of alkali (sodium hydroxide) and constant on amount of BDO formed was investigated. Higher LHSV resulted in lower levels of formation of BDOs, thus the formation of BDOs was minimized when the reaction was operated at higher LHSV (1.5-2.3, Table 4).

### Example 7

Hydrogenolysis of a 40% solution of the polyhydric alcohol glycerol was carried out substantially as described in Example 3 except that 180 mL of Süd Chemie HC-1 catalyst was used. The effect of increasing temperature on BDO formation was investigated. Higher temperatures resulted in formation of greater levels of BDO formation, thus the formation of BDOs was minimized when the reaction was operated at lower temperatures (176-193° C., Table 5).

### Example 8

Product from the hydrogenolysis reactions of Examples 3-7 was purified by distillation to remove BDOs and other reaction products in a product purifier. The pH of hydrogenolysis reaction product was typically in the range of 10.0-14.0. To study the effect of pH, prior to distillation the pH of each reaction product sample was adjusted using concentrated sulfuric acid to produce acidified reaction products. For each experiment, approximately one kilogram of the desired reactor product (“Feed” in Table 6) feed was loaded into a glass vessel and vacuum was applied to reach a pot pressure of approximately 700 millimeters of mercury. Heat was applied using a heating mantle with a variable voltage controller. The sample was allowed to boil and the vapors were condensed and collected separately. The amount of time for this step depended on the desired temperature or the desired quantity of water to be removed; both were experimental variables. The duration of this was usually between two and three hours. The time and maximum pot temperature were recorded for each step. The maximum pot temperature for this step was typically between 180° C. and 190° C. The step is referred to as the initial dewatering step and the distillate product obtained was referred to as the 1^{st} lights cut.

Next, the contents remaining in the still pot (distilland), comprising propylene glycol and other diols, were filtered using a Buchner funnel with Whatman #4 filter paper. The filtration was typically done after the pot temperature had cooled to approximately 95° C. The filter cake was analyzed, and the PG yield loss for this step was determined.

The filtrate was then loaded back into the pot. Vacuum was applied to achieve a pot pressure of approximately 150 millimeters of mercury. Heat was applied to remove the residual water as vapor, which usually took approximately 45 minutes. The maximum pot temperature was typically between 140° C. and 160° C. The vapor (distillate) from this step was condensed and collected. The contents of the pot, comprising propylene glycol and other diols, were then weighed and sampled. This step is referred to as the second dewatering step and the distillate product obtained was referred to as the second lights step.

Finally, the contents of the pot from the second dewatering step, comprising propylene glycol and other diols, were loaded back into the pot and vacuum was applied to reach a pot pressure of 15 millimeters of mercury. Heat was then applied to distill off some of the propylene glycol. The amount of propylene glycol left in the pot was an experimental variable that effected the experimental time and the final pot temperature. The vapors (distillate) from this step were condensed and collected and are referred to as PG cut. The distilland remaining in the still pot (product purifier effluent) was enriched in PG and depleted in butanediols, and is referred to as “Final Bottoms.” The Final Bottoms from the still pot, enriched in PG, were qualitatively observed and described in terms of “flowability” and color.

The propylene glycol yield and accountability were calculated from mass balance data obtained throughout the experiment. The glycerol accountability was also tracked. The PG yield is any PG that was collected in the 1^{st} lights, the second lights, or the PG cut. The PG accountability is the sum of the PG yield, the PG measured in the filter cake, and the PG measured in the final bottoms. The glycerol accountability is all of the measured glycerol that was collected in any sample.

Results from this set of experiments are reported in Table 6. Runs 86 and 89, with pH values lower than 8.0, resulted in higher glycerol accountability and propylene glycol accountability than runs having higher pH values prior to distillation. Consequently, pH adjustment (acidification) of hydrogenolysis product prior to distillation resulted in lower losses of glycerol and higher accountability of propylene glycol. Higher accountabilities in both cases can be interpreted to mean lower degradation of glycerol and propylene glycol in the distillation steps, with accompanying higher levels of propylene glycol recovery. Consequently, it is desirable to run the distillation under conditions which maximize propylene glycol recovery (maximize yield) and higher glycerol accountability (lower degradation of glycerol) to allow for it to be recycled. the content of propylene glycol in product purifier effluent was greater than the content of propylene glycol in the reactor product. In this manner, a three-carbon compound, a four-carbon compound, or any combination thereof could be recovered from the acidified reaction product.

By operating under conditions in described in runs 78, 70, 81, and 84, butanediols were removed from PG and enriched in the distillates, as evidenced by the higher ratio of 2,3 butanediol to PG (g/g) in the first lights from runs 78, 70, 81, and 84, and the second lights from runs 70, 81, and 84. This demonstrated the recovery of a product enriched in four-carbon butanediols from the acidified reaction product. In each run shown in Table 8, the content of 2,3 BDO in the final bottoms was reduced to 0.01 g/100 g solution, or to 0.00 g/100 g solution.

### Example 9

### Effect of Ethanol

Hydrogenolysis of a solution of glycerol and water (25:75) was carried out substantially as described in Example 3 except that 180 mL of Süd Chemie HC-1 catalyst was used. In a second experiment ethanol was added as a solvent with glycerol (glycerol:ethanol:water; 25:55:20). Results presented in Table 7 show that adding ethanol as a solvent to the glycerol feed resulted in lower formation of 2,3 BDO (0.4%) compared to hydrogenolysis without use of a solvent (0.8%).

Consequently as is evident to those skilled in the art suitable conditions exist for hydrogenolysis of sorbitol or glycerol to propylene glycol wherein the yield of propylene glycol is maximized and formation of undesirable side products is minimized. Using the embodiments of this invention one skilled in the art may practice this invention to operate a reactor system and obtain high yields of propylene glycol with low concentrations of other polyhydric alcohols. Alternatively, one skilled in the art may practice this invention to obtain high concentrations of four-carbon polyhydric alcohols.

TABLE 1

| Stream No. | 1 | 4 | 5 |
|---|---|---|---|
| Hydrogen | 98.00 | 99.29 | 60.93 |
| Methane | 0.85 | 0.29 | 16.98 |
| Ethane | 0.85 | 0.14 | 21.17 |
| Methanol | 0.17 | 0.16 | 0.44 |
| Ethanol | 0.13 | 0.12 | 0.48 |
| Pressure, | 1,200.00 | 795.64 | 1,181.14 |
| psia | | | |
| Mass | 2,267.96 | 1,938.83 | 329.12 |
| flow kg/h | | | |

TABLE 2

| Stream No. | 1 | 4 | 5 |
|---|---|---|---|
| Hydrogen | 98.00 | 97.63 | 98.60 |
| Methane | 0.85 | 1.03 | 0.54 |
| Ethane | 0.85 | 1.25 | 0.19 |
| Methanol | 0.17 | 0.04 | 0.38 |
| Ethanol | 0.13 | 0.03 | 0.29 |
| Pressure, | 1,200.00 | 45.73 | 1,148.08 |
| psia | | | |
| Mass | 0.13 | 0.08 | 0.05 |
| flow kg/h | | | |

TABLE 3
Hydrogenolysis of 40% USP Glycerol Feed using a solid phase catalyst.

| | Temperature | | | | | | | | | |
|---|---|---|---|---|---|---|---|---|---|---|
| | distribution in | | | H₂ | NaOH | | | PG | PG | EG |
| Test | Reactor | | | Press. | (%) | | Conversion | Yield | Selectivity | Yield |
| No. | Top | Mid | Bottom | (psi) | ^{w}/_{w} | LHSV | (%) | (%) | (%) | (%) |
| 234 | 196 | 218 | 223 | 1600 | 0.7 | 1.8 | 77.8 | 57 | 88.7 | 3.4 |
| 233 | 195 | 196 | 193 | 1600 | 0.2 | 1.8 | 30 | 27 | 93 | |
| 248 | 183 | 191 | 199 | 1600 | 1 | 2.3 | 47.7 | 36.3 | 92.1 | 1.4 |
| 249 | 184 | 191 | 199 | 1600 | 1 | 1.8 | 56.7 | 42.4 | 90.8 | 1.8 |
| 250 | 185 | 193 | 199 | 1600 | 1 | 1.5 | 63.3 | 47.3 | 90.7 | 2.1 |
| 205 | 178 | 190 | 198 | 1200 | 1.2 | 1.8 | 50 | 38 | 94 | 1.6 |
| 257 | 184 | 195 | 206 | 1600 | 1 | 1.8 | 59.2 | 45.3 | 92.6 | 2.1 |
| 264 | 178 | 190 | 196 | 1600 | 1.9 | 1.6 | 59.3 | 44.3 | 90.3 | 1.9 |
| 261 | 184 | 194 | 200 | 1600 | 1 | 1.5 | 59.4 | 44.4 | 90.4 | 2 |
| 242 | 185 | 194 | 205 | 1600 | 0.7 | 1.8 | 65.2 | 33.2 | 92.2 | 1.5 |
| 199 | 154 | 177 | 194 | 1200 | 1.2 | 1.8 | 67 | 47.9 | 86.6 | 2.2 |
| 262 | 183 | 196 | 202 | 1600 | 1.5 | 1.5 | 67.2 | 49.8 | 89.6 | 2.4 |
| 263 | 181 | 193 | 199 | 1600 | 1.9 | 1.6 | 68.8 | 50.6 | 89.1 | 2.4 |
| 180 | 178 | 191 | 202 | 1200 | 1.1 | 1.0 | 76.7 | 51 | 80.7 | 2.7 |
| 256 | 189 | 206 | 217 | 1600 | 0.8 | 1.8 | 77.1 | 55.9 | 87.6 | 3.1 |
| 254 | 193 | 211 | 223 | 1600 | 1 | 1.8 | 81.2 | 60.8 | 90.6 | 3.8 |
| 255 | 191 | 209 | 221 | 1600 | 0.8 | 1.8 | 86.2 | 52.4 | 73.6 | 3.2 |
| 228 | 193 | 228 | 229 | 1600 | 1.4 | 1.8 | 93.1 | 64 | 83.2 | 4.3 |
| 240 | 188 | 212 | 226 | 1600 | 1.3 | 1.8 | 93 | 63.1 | 82.2 | 3.8 |
| 164 | 183 | 203 | 207 | 1200 | 1.1 | 1 | 94.6 | 68.6 | 87.7 | 3.6 |
| 166 | 188 | 211 | 216 | 1200 | 1.5 | 1 | 95.4 | 47.7 | 60.7 | 2.8 |
| 191 | 165 | 205 | 227 | 1200 | 1.6 | 1.8 | 96.4 | 55.4 | 69.6 | 3.6 |

| | | | | |
|---|---|---|---|---|
| | | | 1,2 | |
| | | | BDO | Butanediols and other diols (g/100 g |
| Lactic | EtOH | MeOH | & | solution) |

| Test | Yield | Yield | Yield | 2,3- | 1-2 | 1-3 | 1-4 | 2-3 | 2-4 |
|---|---|---|---|---|---|---|---|---|---|
| No. | (%) | (%) | (%) | BDO | BDO | BDO | BDO | BDO | PeDO |
| 234 | 1 | 0.1 | 1.5 | 0.13 | 0 | 0 | 0 | 0.03 | 0 |
| 233 | | | | 0.46 | 0.01 | 0 | 0 | 0.04 | 0 |
| 248 | 0.5 | 0 | 0.7 | 0.27 | 0 | 0 | 0 | 0.04 | 0 |
| 249 | 0.6 | 0 | 0.9 | 0.29 | 0 | 0 | 0 | 0.05 | 0 |
| 250 | 0.7 | 0.1 | 0.9 | 0.58 | 0.02 | 0 | 0 | 0.09 | 0.01 |
| 205 | 1 | 0.1 | 0 | 1.23 | 0.03 | 0 | 0 | 0.16 | 0.03 |
| 257 | 0.7 | 0.1 | 1 | 0.28 | 0 | 0.02 | 0 | 0.05 | 0 |
| 264 | 0.9 | 0 | 1 | 0.28 | 0 | 0.01 | 0 | 0.05 | 0 |
| 261 | 0.7 | 0.1 | 1.1 | 0.28 | 0 | 0 | 0 | 0.05 | 0 |
| 242 | 0.5 | 0 | 0.7 | 2.28 | 0.05 | 0 | 0 | 0.26 | 0.04 |
| 199 | 1.1 | 0.3 | 1 | 2.29 | 0.07 | 0 | 0 | 0.38 | 0.08 |
| 262 | 0.9 | 0.1 | 1.3 | 0.40 | 0 | 0 | 0 | 0.08 | 0.01 |
| 263 | 1.1 | 0.1 | 1.2 | 0.49 | 0 | 0.01 | 0 | 0.1 | 0.01 |
| 180 | 1.5 | 0.7 | 1.3 | 3.18 | 0.1 | 0 | 0 | 0.57 | 0.14 |
| 256 | 1 | 0.2 | 1.5 | 0.75 | 0 | 0.04 | 0 | 0.17 | 0.02 |
| 254 | 1.2 | 0.3 | 1.8 | 1.18 | 0 | 0.06 | 0 | 0.29 | 0.04 |
| 255 | 1 | 0.2 | 1.6 | 1.36 | 0 | 0.06 | 0 | 0.29 | 0.04 |
| 228 | 1.9 | 1 | 0.6 | 3.14 | 0.14 | 0 | 0 | 0.69 | 0.14 |
| 240 | 1.8 | 1 | 1.7 | 4.68 | 0.19 | 0 | 0 | 1.05 | 0.23 |
| 164 | 1.8 | 0.6 | 1.7 | 2.59 | 0.12 | 0 | 0 | 0.61 | 0.12 |
| 166 | 1.5 | 0.7 | 2 | 4.65 | 0.12 | 0 | 0 | 0.81 | 0.19 |
| 191 | 2.8 | 2.1 | 1.4 | 7.82 | 0.23 | 0 | 0 | 1.65 | 0.43 |

TABLE 4

| | Temperature | | | | | | | | PG | 1,2 | Butanediols and other diols | | | | | | |
|---|---|---|---|---|---|---|---|---|---|---|---|---|---|---|---|---|---|
| | distribution in Reactor | | | H₂ | NaOH | | Con- | PG | Selec- | BDO & | (g/100 g solution) | | | | | | |
| Test | | | Bot- | Press. | (%) | | version | Yield | tivity | 2,3- | 1-2 | 1-3 | 1-4 | 2-3 | 2-4 | 1-3 | 1-5 |
| No. | Top | Mid | tom | (psi) | ^{w}/_{w} | LHSV | (%) | (%) | (%) | BDO | BDO | BDO | BDO | BDO | PeDO | PrDO | PeDO |
| 25 | 167 | 170 | 168 | 1000 | 0.5 | 0.5 | 28 | 21 | 92 | 0.12 | 0 | 0 | 0.01 | 0.01 | 0 | 0 | 0 |
| 5 | 150 | 166 | 169 | 1000 | 2.4 | 0.6 | 75 | 57 | 93 | 0.18 | 0 | 0 | 0 | 0.04 | 0 | 0 | 0 |
| 6 | 150 | 166 | 169 | 1000 | 0.5 | 2.0 | 14 | 10 | 88 | 0.00 | 0 | 0 | 0.01 | 0 | 0 | 0 | 0 |
| 27 | 155 | 170 | 171 | 1000 | 2.5 | 1.8 | 32 | 24 | 92 | 0.41 | 0 | 0 | 0.01 | 0.04 | 0 | 0 | 0 |
| 28 | 205 | 213 | 204 | 1600 | 0.5 | 0.6 | 82 | 59 | 88 | 0.92 | 0.05 | 0 | 0 | 0.22 | 0.03 | 0 | 0 |
| 26 | 206 | 209 | 203 | 1000 | 2.4 | 0.6 | 97 | 51 | 64 | 8.89 | 0.32 | 0 | 0 | 1.99 | 0.72 | 0 | 0 |
| 8 | 179 | 202 | 210 | 1600 | 0.5 | 2.0 | 58 | 43 | 91 | 0.46 | 0.01 | 0 | 0 | 0.08 | 0 | 0 | 0 |
| 9 | 171 | 212 | 208 | 1000 | 2.5 | 1.8 | 97 | 60 | 76 | 6.07 | 0.2 | 0 | 0.01 | 1.55 | 0.43 | 0 | 0 |

TABLE 5

| | Temperature | | | | | | | | PG | | 1,2 | Butanediols and other diols | | | | |
|---|---|---|---|---|---|---|---|---|---|---|---|---|---|---|---|---|
| | distribution in Reactor | | | H₂ | NaOH | | Con- | PG | Selec- | EG | BDO & | (g/100 g solution) | | | | |
| Test | | | Bot- | Press. | (%) | | version | Yield | tivity | Yield | 2,3- | 1-2 | 1-3 | 1-4 | 2-3 | 2-4 |
| No. | Top | Mid | tom | (psi) | ^{w}/_{w} | LHSV | (%) | (%) | (%) | (%) | BDO | BDO | BDO | BDO | BDO | PeDO |
| 283 | 176 | 193 | 176 | 1600 | 0.8 | 1.01 | 83.7 | 56 | 80.9 | 4.37 | 0.22 | 0.02 | ND | ND | 0.03 | ND |
| 284 | 188 | 204 | 188 | 1600 | 0.8 | 1 | 92.4 | 61 | 79.9 | 5.3 | 0.49 | 0.04 | ND | ND | 0.08 | ND |
| 285 | 195 | 216 | 195 | 1600 | 0.8 | 1.02 | 97.5 | 60.1 | 74.6 | 5.2 | 0.66 | 0.05 | ND | ND | 0.11 | ND |

TABLE 6

| | | PG | | | | | | | | Final |
|---|---|---|---|---|---|---|---|---|---|---|
| | | Distillation | | | | Feed | 1st lights | 2nd lights | PG Cut | Bottoms |
| | Feed | final pot | PG | PG | Glycerol | 2,3BDO/PG | 2,3BDO/PG | 2,3BDO/PG | 2,3BDO/PG | 2,3BDO/PG |
| Run | pH | Temp (C.) | accountability | distilled | accountability | (g/g) | (g/g) | (g/g) | (g/g) | (g/g) |
| 78 | 12.49 | 295 | 95.9 | 87.7 | 13.5 | 0.02 | 0.18 | 0.02 | 0.02 | 0.01 |
| 70 | 10.64 | 290 | 96.6 | 73.8 | 66.2 | 0.02 | 0.14 | 0.04 | 0.02 | 0.01 |
| 81 | 4.55 | 368 | 97.1 | 87.4 | 50.4 | 0.02 | 0.14 | 0.04 | 0.02 | 0.00 |
| 84 | 7.66 | 368 | 98.1 | 88.8 | 69.7 | 0.02 | 0.10 | 0.05 | 0.02 | 0.01 |
| 86 | 5.85 | 206 | 97.9 | 30.3 | 92.8 | | | | | |
| 88 | 12.6 | 176 | 77.5 | 40.2 | 78.5 | 0.00 | 0 | 0.01 | 0.00 | 0.00 |
| 89 | 7.69 | 206 | 103.5 | 73.6 | 97.4 | 0.00 | 0 | 0.01 | 0.00 | 0.00 |

TABLE 7

| | without Ethanol | with Ethanol added |
|---|---|---|
| Temperature (deg C.) | 215 | 181 |
| Pressure (psig) | 1600 | 1500 |
| NaOH (%, w/w) | 0.68 | 0.35 |
| LHSV | 1.65 | 1.8 |
| Conversion (%) | 81.2 | 79 |
| PG Yield (%) | 59.2 | 59.8 |
| PG Selectivity (%) | 88.3 | 91.5 |
| 2,3 BDO (%, PG Basis) | 0.8 | 0.4 |
| Productivity (g PG/L catalyst/ | 264.2 | 238.3 |
| Hr) | | |

1. A system for treating glycerol or sorbitol comprising:
a reactor operably configured to convert the glycerol or the sorbitol to a reaction product comprising propylene glycol and a butanediol selected from the group consisting of 1,2-butanediol, 1,3-butanediol, 1,4-butanediol, 2,3-butanediol, and combinations of any thereof;
a first conduit comprising the glycerol or the sorbitol operably connected to the reactor;
a product purifier operably configured to remove the butanediol from the reaction product; and
a second conduit comprising the reaction product operably connected to the reactor and the product purifier.

2. The system of claim 1, further comprising a third conduit comprising a product purifier effluent operably connected to the product purifier.

3. The system of claim 2, wherein a content of the butanediol in the reaction product is greater than the content of butanediol in the product purifier effluent.

4. The system of claim 1, wherein the content of butanediol in the reaction product is 0.04 to 2.31 grams/100 grams of solution.

5. The system of claim 1, wherein a content of the butanediol in product purifier effluent is less than 0.02%.

6. The system of claim 1, further comprising means for purifying the glycerol before the glycerol enters the first conduit.

7. The system of claim 6, wherein the means for purifying the glycerol purifies the glycerol by an act selected from the group consisting of placing a solution of glycerol in an electro-dialysis apparatus, distilling a solution of glycerol, subjecting a solution of glycerol to ion exchange, and combinations of any thereof, thus removing salt impurities from the glycerol.

8-9. (canceled)

10. A process for converting a polyhydric alcohol into a three-carbon compound, a four-carbon compound or a combination thereof, the process comprising:
combining the polyhydric alcohol with hydrogen, thus forming a reaction mixture;
heating the reaction mixture;
placing the reaction mixture in contact with a catalyst, thus forming a reaction product;
acidifying the reaction product; and
removing the three-carbon compound, the four-carbon compound, or any combination thereof from the acidified reaction product.

11. The process of claim 10, wherein the polyhydric alcohol comprises glycerol, sorbitol or a combination thereof.

12. The process of claim 10, wherein the four-carbon compound comprises a compound selected from the group consisting of 1,2-butanediol, 1,3-butanediol, 1,4-butanediol, 2,3-butanediol, and combinations of any thereof.

13-16. (canceled)

17. The process of claim 10, wherein the three-carbon compound product comprises propylene glycol.

18. The process of claim 10, further comprising purifying the three-carbon compound, the four carbon compound or the combination thereof.

19. The process of claim 18, wherein the ratio of three-carbon product to four-carbon product after the purifying is different from the ratio of three-carbon product to four-carbon product before the purifying.

20. The process of claim 18, wherein the purifying comprises an act selected from the group consisting of ion exclusion, electro-dialysis, filtration, distillation, ion exchange, and any combinations thereof.

21. (canceled)

22. A composition enriched in a four-carbon compound produced by the process of claim 10.

23. (canceled)

24. A composition enriched in a three-carbon compound produced by the process of claim 10.

25-31. (canceled)

32. The process of claim 10, wherein the polyhydric alcohol is glycerol, sorbitol or a combination thereof, the process further comprising:
separating a vapor, a gas or a combination thereof from the reaction product;
wherein the three-carbon compound is propylene glycol and the four-carbon compound is ethylene glycol.

33. (canceled)

34. The process of claim 32, wherein
the catalyst is a heterogeneous catalyst selected from the group consisting of rhenium, nickel, and a combination thereof; and
the catalyst is embedded in an activated carbon matrix.

35-38. (canceled)

39. A composition comprising a bio-based propylene glycol having a measurable content of butanediol, wherein the content of butanediol is less than 0.25%.

40. The composition of claim 39, wherein the butanediol content is less than 0.1%.

41-45. (canceled)
